(19) United States
(12) Patent Application Publication
(10) Pub. No.: US 2024/0171009 A1
(43) Pub. Date: May 23, 2024

(54) TRANSFORMER IMPLANT FOR INDUCTIVE POWER SUPPLY TO IMPLANTED MEDICAL DEVICE

(71) Applicant: InvivoPower AB, MÖLNDAL (SE)

(72) Inventor: Bengt Bern, MÖLNDAL (SE)

(21) Appl. No.: 18/500,176

(22) Filed: Nov. 2, 2023

(30) Foreign Application Priority Data
Nov. 18, 2022 (SE) 2251354-3

(51) Int. Cl. H02J 50/10; A61M 60/873; H01F 27/24; H01F 27/28

(52) U.S. Cl. CPC H02J 50/10; A61M 60/873; H01F 27/24; H01F 27/28; A61N 1/3787

(57) ABSTRACT

A transformer implant comprising a ring-shaped transformer core; and elastomeric material embedding the ring-shaped transformer core, in such a way that an opening through the ring-shaped core is provided. The elastomeric material defines a base surface of the transformer implant and an upper surface of the transformer implant, the base surface and the upper surface meeting each other along an edge line of the transformer implant defining a maximum lateral extension of the transformer implant. The ring-shaped transformer core is arranged in a standing configuration, such that the ring-shaped transformer core has a maximum vertical extension along a first straight line and a maximum lateral extension along a second straight line. A lateral distance, in a direction parallel to the second straight line, from the first straight line to the edge line of the transformer implant at least corresponds to the maximum vertical extension of the ring-shaped transformer.

### FIELD OF THE INVENTION

The present invention relates to a transformer implant, and to a medical system comprising the transformer implant.

### BACKGROUND OF THE INVENTION

Medical devices having one or more implantable units, generally referred to as implantable medical devices, have provided a wide range of benefits to patients over recent decades. In particular, devices such as implantable hearing aids, implantable pacemakers, defibrillators, eye implants, retina implants, heart pumps, ventricular assist devices, total artificial hearts, drug delivery systems, gastric implants, nerve stimulators, brain stimulators, functional electrical stimulation devices, such as cochlear prostheses, organ assist or replacement devices, and other partially or completely-implanted medical devices, have been successful in performing life-saving and/or lifestyle enhancement functions for a number of years.

As such, the types of implantable devices and the range of functions performed thereby have increased over the years. For example, many such implantable medical devices often include one or more instruments, apparatuses, sensors, processors, controllers or other functional mechanical, electrical or electronic components that are permanently or temporarily implanted in a patient to perform diagnosis, prevention, monitoring, treatment or management of a disease or injury or symptom thereof, or to investigate, replace or modify of the anatomy or of a physiological process. Many of these implantable components receive power and/or receive data and/or transmit data over a wireless transcutaneous link from and/or to external units that are part of, or operate in conjunction with, the implantable unit.

The wireless transcutaneous link is conventionally realized as an inductive link, with an external unit comprising a transmitter winding and an implantable unit comprising a receiver winding. Typically, the receiver winding is implanted below the skin, and the transmitter winding is attached to the patient's skin opposite to the implanted receiver winding such that the two windings are in parallel planes on both sides (external and implantable positions) of the skin. These systems are typically referred as TET links (TET-Transcutaneous Energy Transfer). For TET links it is rather difficult to fixate and position the transmitter winding to the skin of a patient. Gluing solutions and special vests to fixate/position the transmitter winding have been tried but have been associated with problems especially when the patient has been sleeping. For life sustaining applications like heart pumps, ventricular assist devices or total artificial hearts, this fixation and positioning is very critical. If the transmitter winding falls off or if it is in the wrong position, the transcutaneous power transfer is affected and could in worst case be life threatening for the patient. Therefore, TET systems for life sustaining applications are almost always implemented with an implantable battery. If the transmitter winding falls off or if it is in the wrong position, the implantable battery will continue to supply power to the implantable unit. The implantable unit could, for instance, be a heart pump. However, implantable batteries give rise to a new problem. Today's battery technology has a limited number of recharging cycles. For an example if the battery recharging is limited to one thousand charging cycles and the patient charges the implantable battery two times a day, the battery needs to be replaced every one and a half years. Frequent replacement of the implantable battery requires costly surgery, increases the risk for infection for the patient and reduces the quality of life for the patient due to repeated hospital stays. Therefore there is a need for an improved transcutaneous energy supply system, reducing risks and discomfort for the patient.

An improved medical system, providing for improved energy transfer between an external unit and an internal unit is described in SE 543 180 and SE 543 181. In this medical system, a ring-shaped transformer core implant is arranged under the skin of the patient and an externally accessible passage is formed through the ring-shaped transformer core. It would be desirable to provide an improved transformer implant, in particular offering improved implantability and an improved patient experience of the medical system comprising the transformer implant.

### SUMMARY

It is an object of the present invention to provide an improved transformer implant, in particular offering improved implantability and an improved patient experience of the medical system comprising the transformer implant.

According to an aspect of the present invention, it is therefore provided a transformer implant for inductive transfer of power from an external unit of a medical system, via the transformer implant, to an internal unit implanted into a body of a patient, the transformer implant comprising: a ring-shaped transformer core; an internal transformer winding around the ring-shaped transformer core; and elastomeric material embedding the ring-shaped transformer core, in such a way that an opening through the ring-shaped core is maintained to accommodate an external winding around the ring-shaped transformer core following implantation, wherein the elastomeric material defines a base surface of the transformer implant to be arranged facing away from an inside of the skin of the patient when implanted, and an upper surface of the transformer implant to be arranged facing the inside of the skin of the patient when implanted, the base surface and the upper surface meeting each other along an edge line of the transformer implant defining a maximum lateral extension of the transformer implant, wherein the ring-shaped transformer core is arranged in a standing configuration in relation to the base surface, such that the ring-shaped transformer core has a maximum vertical extension along a first straight line and a maximum lateral extension along a second straight line, and wherein a lateral distance, in a direction parallel to the second straight line, from the first straight line to the edge line of the transformer implant at least corresponds to the maximum vertical extension of the ring-shaped transformer.

The present invention is based on the realization that improved implantability and an improved patient experience, with maintained electrical properties, can be achieved by embedding the ring-shaped transformer core of the implant in elastomeric material in such a way that an opening through the ring-shaped core is provided to accommodate an external winding around the ring-shaped transformer core following implantation. Further, by shaping the embedding elastomeric material such that the width of the embedded implant at least corresponds to the two times the height of the embedded implant, the effect of an impact by a hard object on the skin of the patient can be reduced, and the pressure-distribution across the surface of the skin covering the implant can be made more even, as compared to a corresponding implant without embedding elastomeric material. These effects obtainable using the transformer implant according to aspects of the present invention are foreseen to improve the comfort for patients of a medical system including the transformer implant, and to reduce the risk of tissue damage.

The transformer implant according to various embodiments of the present invention may advantageously be included in a medical system, further comprising an internal unit implantable into a body of a patient; and an external unit to remain outside the body of the patient, the external unit comprising external cabling configured to allow formation of an external winding around the ring-shaped transformer core of the transformer implant, and power supply circuitry coupled to the external cabling and configured to supply power to the internal unit via the ring-shaped transformer core of the transformer implant.

In summary, the present invention relates to a transformer implant comprising a ring-shaped transformer core; and elastomeric material embedding the ring-shaped transformer core, in such a way that an opening through the ring-shaped core is provided. The elastomeric material defines a base surface of the transformer implant and an upper surface of the transformer implant, the base surface and the upper surface meeting each other along an edge line of the transformer implant defining a maximum lateral extension of the transformer implant. The ring-shaped transformer core is arranged in a standing configuration, such that the ring-shaped transformer core has a maximum vertical extension along a first straight line and a maximum lateral extension along a second straight line. A lateral distance, in a direction parallel to the second straight line, from the first straight line to the edge line of the transformer implant at least corresponds to the maximum vertical extension of the ring-shaped transformer.

### BRIEF DESCRIPTION OF THE DRAWINGS

These and other aspects of the present invention will now be described in more detail, with reference to the appended drawings showing an example embodiment of the invention, wherein:

FIG. 1 illustrates an exemplary medical system comprising a transformer implant according to an example embodiment of the present invention;

FIG. 2 is an enlarged partial illustration of the medical system in FIG. 1, focusing on the implanted transformer implant;

FIGS. 3A-B are schematic cross-section views of the implanted transformer implant in FIG. 2;

FIGS. 4A-B are perspective views of a transformer implant according to an example embodiment of the present invention;

FIG. 5 is a top view of the transformer implant in FIGS. 4A-B;

FIG. 6 is a first cross-section view of the transformer implant in FIGS. 4A-B; and

FIG. 7 is a second cross-section view of the transformer implant in FIGS. 4A-B.

### DETAILED DESCRIPTION OF EXAMPLE EMBODIMENTS

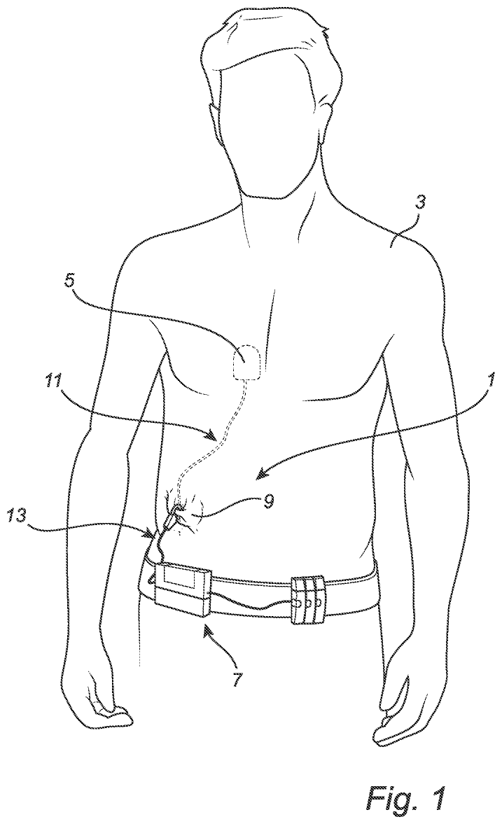

FIG. 1 illustrates an exemplary medical system 1 when in use by a patient 3. The medical system comprises an internal unit 5, an external unit 7, and the transformer implant 9 according to embodiments of the present invention. The internal unit 5 is coupled to the transformer implant 9 by internal cabling 11, and the external unit 7 is coupled to the transformer implant 9 by external cabling 13. The transformer implant 9, the internal cabling 11, and the external cabling 13 provide an efficient inductive power link for providing power from the external unit 7 to the internal unit 5 for operation of the internal unit 5.

In FIG. 1, the internal unit 5 is schematically indicated as being a heart pump or an artificial heart—with or without a rechargeable battery. It should be noted that these are only illustrative examples of internal units that would benefit from use of the inductive power link enabled by the transformer implant according to embodiments of the present invention. Examples of other internal units may include a cochlear implant, an auditory transmodiolar implant, an auditory brainstem implant, a bone conduction hearing aid, a middle ear implant, an artificial pacemaker, a blood pumping impeller, a ventricular assist device (VAD), a total artificial heart, an eye implant or retina implant, a nerve stimulator, a deep brain stimulator, a drug delivery system, a brain computer interface system, a cardioverter defibrillator, a gastric stimulator, a brain computer interface system or a rechargeable battery, or combinations thereof.

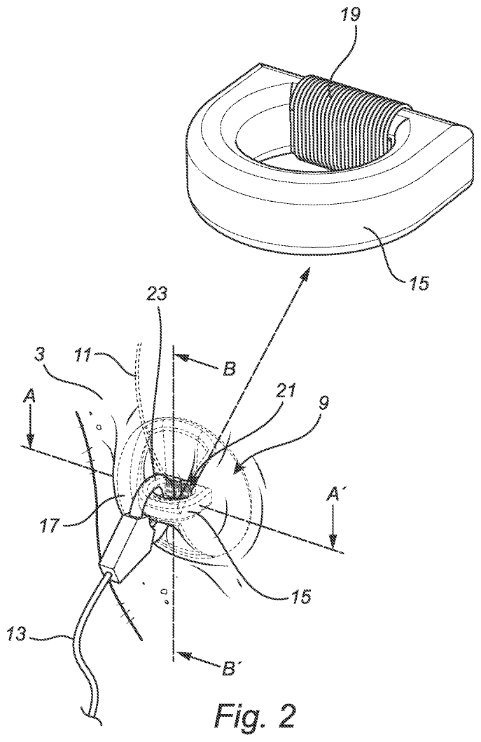

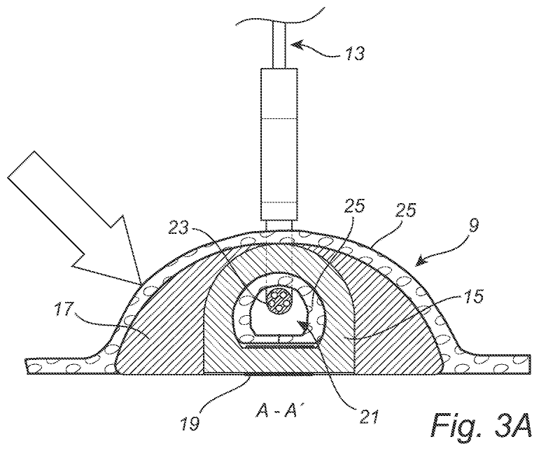

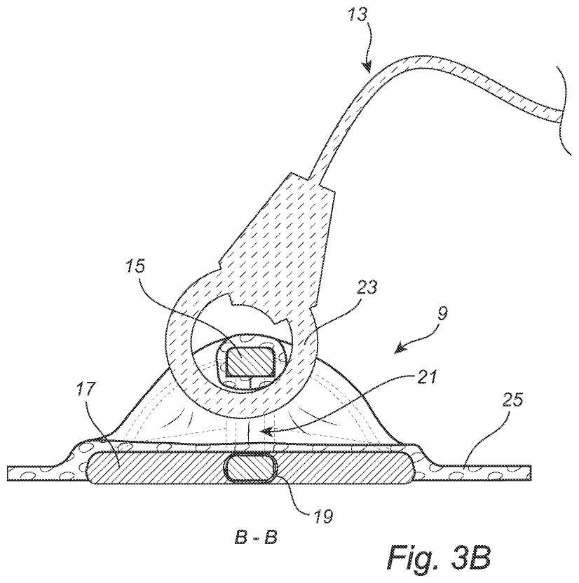

FIG. 2 is an enlarged partial illustration of the medical system 1 in FIG. 1, focusing on the implanted transformer implant 9. FIG. 3A is a cross-section view of the section taken along the line A-A′ in FIG. 2, and FIG. 3B is a cross-section view of the section taken along the line B-B′ in FIG. 2. Referring to FIG. 2, and FIGS. 3A-B, the transformer implant 9 comprises a ring-shaped transformer core 15, and elastomeric material 17 embedding the ring-shaped transformer core 15 and an internal winding 19 around the ring-shaped transformer core 15. The above-mentioned internal cabling 11 of the medical system 1 is connected to the internal winding 19. The elastomeric material 17 embeds the ring-shaped transformer core 15 in such a way that an opening 21 through the ring-shaped transformer core 15 is provided to accommodate an external winding 23 around the ring-shaped transformer core 15 after implantation. The above-mentioned external cabling 13 is connected to the external winding 23, so that an inductive power link is formed from the external cabling 13 to the internal cabling 11, via the external winding 23, the ring-shaped transformer core 15, and the internal winding 19. As is schematically shown in FIG. 2 and FIGS. 3A-B, the skin 25 of the patient 3 is present inside the opening 21 through the ring-shaped transformer core 15, and around the outline of the transformer implant 9. The ring-shaped transformer core 15 is inherently hard. By embedding the ring-shaped transformer core 15 in the elastomeric material 17, the effect of an impact—represented by the block arrow in FIG. 3A—by a hard object on the skin 25 of the patient 3 can be reduced. In addition, the pressure-distribution across the surface of the skin 25 covering the transformer implant 9 can be made more even.

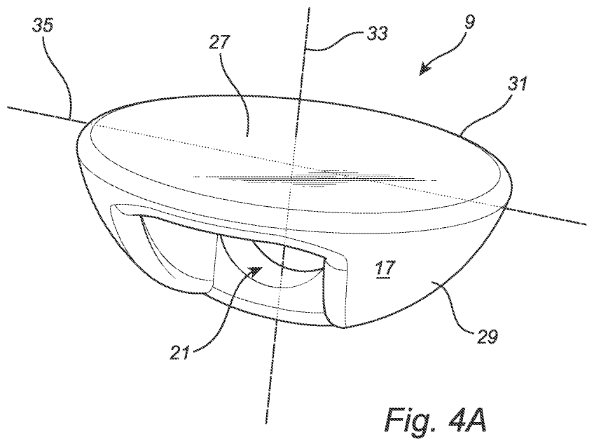

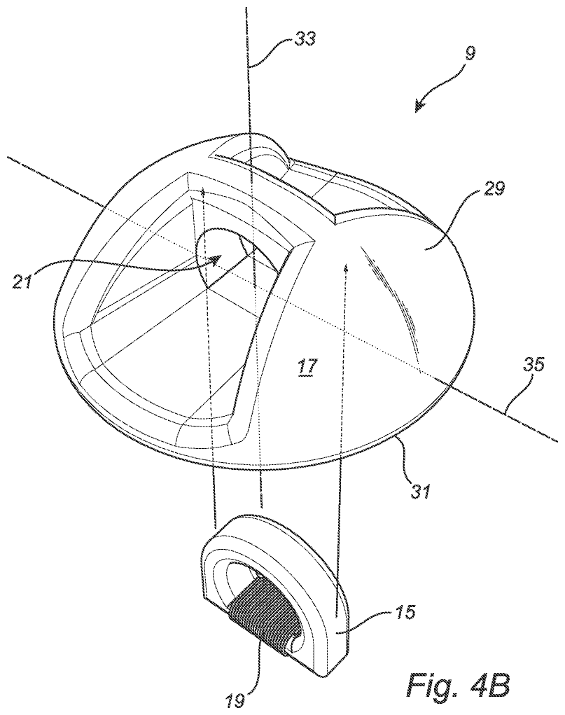

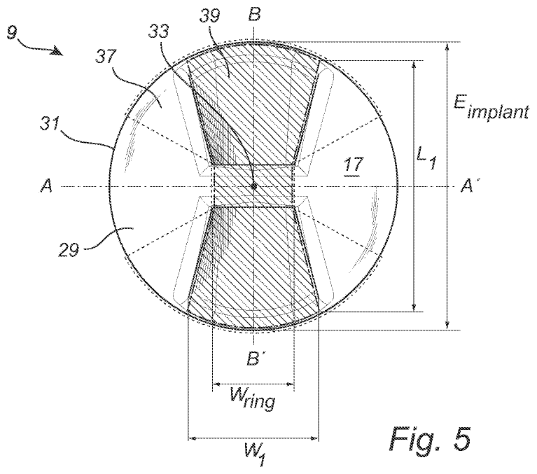

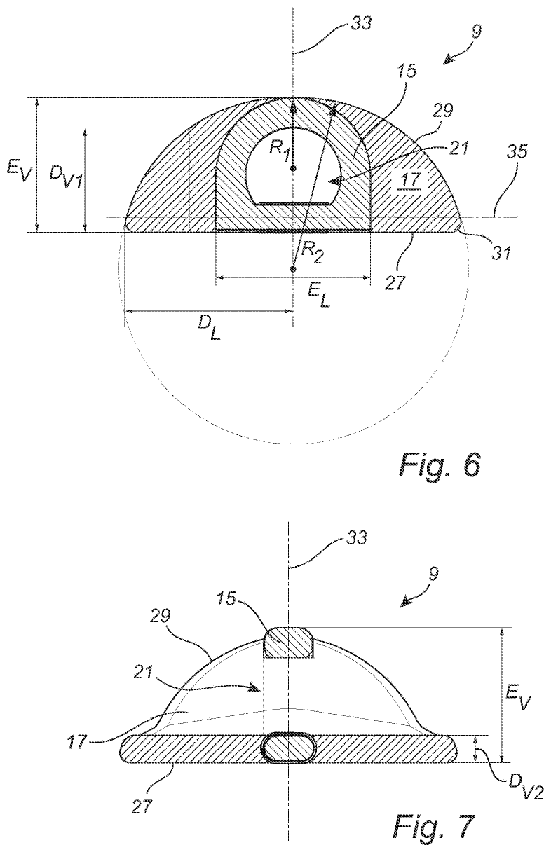

FIG. 4A-B is a perspective views of a transformer implant 9 according to an example embodiment of the present invention, as seen generally from below (FIG. 4A) and from above (FIG. 4B). FIG. 5 is a top view of the transformer implant 9 in FIGS. 4A-B, FIG. 6 is a first cross-section view (A-A′) of the transformer implant 9 in FIG. 4, and FIG. 7 is a second cross-section view (B-B′) of the transformer implant 9 in FIGS. 4A-B.

The elastomeric material 17 defines a base surface 27 of the transformer implant 9, to be arranged facing away from the inside of the skin of the patient when implanted (compare FIG. 2 and FIGS. 3A-B), and an upper surface 29 to be arranged facing the inside of the skin of the patient when implanted. The base surface 27 and the upper surface 29 meet each other along an edge line 31 of the transformer implant 9, defining a maximum lateral extension of the transformer implant 9. The ring-shaped transformer core 15 is arranged in a standing configuration in relation to the base surface 27, such that the ring-shaped transformer core has a maximum vertical extension E_{V} along a first straight line 33, and a maximum lateral extension E_{L} along a second straight line 35. A lateral distance D_{L} in a direction parallel to the second straight line 35, from the first straight line 33 to the edge line 31 of the transformer implant 9 at least corresponds to the maximum vertical extension E_{V} of the ring-shaped transformer 15.

According to embodiments, a vertical distance D_{V1} between the base surface 27 of the transformer implant 9 and the upper surface 29 of the transformer implant may decrease with increasing lateral distance from the first straight line 33. In particular, with reference to, for example, FIG. 6, the vertical distance D_{V1} may decrease monotonically with increasing lateral distance from the first straight line 33. Advantageously, the vertical distance D_{V1} between the base surface 27 of the transformer implant 9 and the upper surface 29 of the transformer implant 9 may decrease monotonically with increasing lateral distance from the first straight line 33, all the way from the first straight line 33 to the edge line 31 of the transformer implant 9. In these configurations, the desired impact protection of the skin 25 of the patient 3 can be achieved without requiring unnecessary additional tensioning in the skin during the implantation procedure.

Furthermore, a maximum vertical distance between the base surface 27 of the transformer implant 9 and the upper surface 29 of the transformer implant 9 may be at least 90% of a maximum vertical extension of the transformer implant 9, including the ring-shaped transformer core 15.

With main reference now to the top view in FIG. 5 and the cross-section view in FIG. 7, example embodiments of the transformer implant 9 may comprise a first portion 37 having a lateral extent defined by the edge line 31 of the transformer implant 9 and vertical projections of all straight lines being perpendicular to the first straight line 33 and passing through the opening 21 of the ring-shaped transformer core 15. At least in a sub-portion 39 (an exemplary sub-portion is indicated by hatched lines in FIG. 5), a maximum vertical distance D_{V2} between the base surface 27 of the transformer implant 9 and the upper surface 29 of the transformer implant 9 is less than one half of the maximum vertical extension E_{V} of the ring-shaped transformer core 15. Through this configuration, the impact of the embedding elastomeric material 17 on the implantation procedure, as well as on the insertion of the external winding 23 (FIG. 2 and FIGS. 3A-B) can be at least acceptable.

In example configurations, the above-mentioned sub-portion 39 may be laterally oblong with a (maximum) width W₁ and a (maximum) length L₁, and the width W₁ may be at least one half of a maximum width Wring of the ring opening of the ring-shaped transformer core 15. Furthermore, the length L₁ of the sub-portion 39 may correspond to at least one half of a maximum lateral extension Eᵢₘₚₗₐₙₜ, in a length direction of the sub-portion 39.

According to embodiments, the vertical distance D_{V1} between the base surface 27 and the upper surface 29 may decrease with increasing lateral distance from the first straight line 33, everywhere outside the first portion 37 of the transformer implant 9. As mentioned further above, this decrease may be monotonical, and it may also be continuous.

According to embodiments, the ring-shaped transformer core 15 may exhibit a first radius R₁ of curvature in a point defined by the maximum vertical extension E_{V} of the ring-shaped transformer core 15 and a plane (the plane of the paper in FIG. 6) including the first straight line 33 and the ring opening 21 of the ring-shaped transformer core, and the transformer implant 9 may exhibit a second radius R₂ of curvature in the plane including the first straight line and the ring opening of the ring-shaped transformer core, the second radius R₂ of curvature being greater than the first radius R₁ of curvature.

As is evident from the illustrations in the figures, there is no thick layer of the elastomeric material 17 at the top of the ring-shaped transformer core 15. A reason for this is to not increase the requirement on skin lengthening in the cross-section shown in FIG. 3B, where the skin 25 should be wrapped around the ring opening of the ring-shaped transformer core 15. Through the above-described configuration, with a greater radius R₂ of curvature of the transformer implant 9 (by means of the shape of the embedding elastomeric material 17) than the radius R₁ of curvature of the ring-shaped transformer core 15, the occurrence and/or effect of unwanted interactions between any object and the skin at the top of the ring-shaped transformer core 15 can be reduced.

The elastomeric material 17 embedding the ring-shaped transformer core 15 may advantageously exhibit a shore A hardness that is less than 50, preferably less than 40. Furthermore, the elastomeric material may include silicone, which is a proven material that is suitable for implantation.

In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

What is claimed is:

1. A transformer implant for inductive transfer of power from an external unit of a medical system, via the transformer implant, to an internal unit implanted into a body of a patient, the transformer implant comprising:
a ring-shaped transformer core;
an internal transformer winding around the ring-shaped transformer core; and
elastomeric material embedding the ring-shaped transformer core, in such a way that an opening through the ring-shaped core is provided to accommodate an external winding around the ring-shaped transformer core following implantation,
wherein the elastomeric material defines a base surface of the transformer implant to be arranged facing away from an inside of the skin of the patient when implanted, and an upper surface of the transformer implant to be arranged facing the inside of the skin of the patient when implanted, the base surface and the upper surface meeting each other along an edge line of the transformer implant defining a maximum lateral extension of the transformer implant,
wherein the ring-shaped transformer core is arranged in a standing configuration in relation to the base surface, such that the ring-shaped transformer core has a maximum vertical extension along a first straight line and a maximum lateral extension along a second straight line, and
wherein a lateral distance, in a direction parallel to the second straight line, from the first straight line to the edge line of the transformer implant at least corresponds to the maximum vertical extension of the ring-shaped transformer.

2. The transformer implant according to claim 1, wherein a vertical distance between the base surface of the transformer implant and the upper surface of the transformer implant decreases with increasing lateral distance from the first straight line.

3. The transformer implant according to claim 2, wherein the vertical distance between the base surface of the transformer implant and the upper surface of the transformer implant decreases monotonically with increasing lateral distance from the first straight line.

4. The transformer implant according to claim 3, wherein the vertical distance between the base surface of the transformer implant and the upper surface of the transformer implant decreases monotonically with increasing lateral distance from the first straight line, from the first straight line to the edge line of the transformer implant.

5. The transformer implant according to claim 2, wherein:
the transformer implant comprises a first portion, having a lateral extent defined by the edge line of the transformer implant and vertical projections of all straight lines being perpendicular to the first straight line and passing through a ring opening of the ring-shaped transformer core; and
at least in a sub-portion of the first portion, a maximum vertical distance between the base surface of the transformer implant and the upper surface of the transformer implant is less than one half of the maximum vertical extension of the ring-shaped transformer core.

6. The transformer implant according to claim 5, wherein:
the sub-portion of the first portion is laterally oblong with a width and a length; and
the width of the sub-portion is at least one half of a maximum width of the ring opening of the ring-shaped transformer core.

7. The transformer implant according to claim 6, wherein the length of the sub-portion corresponds to at least one half of a maximum lateral extension of the transformer implant, in a length direction of the sub-portion.

8. The transformer implant according to claim 5, wherein the vertical distance between the base surface of the transformer implant and the upper surface of the transformer implant decreases with increasing lateral distance from the first straight line, everywhere outside the first portion of the transformer implant.

9. The transformer implant according to claim 5, wherein:
the ring-shaped transformer core exhibits a first radius of curvature in a point defined by the maximum vertical extension of the ring-shaped transformer core and a plane including the first straight line and the ring opening of the ring-shaped transformer core; and
the transformer implant exhibits a second radius of curvature in the plane including the first straight line and the ring opening of the ring-shaped transformer core, the second radius of curvature being greater than the first radius of curvature.

10. The transformer implant according to claim 1, wherein a maximum vertical distance between the base surface of the transformer implant and the upper surface of the transformer implant is at least 90% of a maximum vertical extension of the transformer implant.

11. The transformer implant according to claim 1, wherein the elastomeric material exhibits a Shore A hardness less than 50.

12. The transformer implant according to claim 1, wherein the elastomeric material includes silicone.

13. A medical system comprising:
an internal unit implantable into a body of a patient;
the transformer implant according to claim 1; and
an external unit to remain outside the body of the patient, the external unit comprising external cabling configured to allow formation of an external winding around the ring-shaped transformer core of the transformer implant, and power supply circuitry coupled to the external cabling and configured to supply power to the internal unit via the ring-shaped transformer core of the transformer implant.

14. The medical system according to claim 13, wherein the internal unit is a cochlear implant, an auditory transmodiolar implant, an auditory brainstem implant, a bone conduction hearing aid, a middle ear implant, an artificial pacemaker, a blood pumping impeller, a ventricular assist device (VAD), a total artificial heart, an eye implant or retina implant, a nerve stimulator, a deep brain stimulator, a drug delivery system, a brain computer interface system, a cardioverter defibrillator, a gastric stimulator, a brain computer interface system or a rechargeable battery.
